(19) United States
(12) Patent Application Publication
(10) Pub. No.: US 2003/0207820 A1
(43) Pub. Date: Nov. 6, 2003

(54) Antibacterial compounds with improved pharmacokinetic profiles

(76) Inventors: Richard F. Clark, Gurnee, IL (US); Stevan Djuric, Libertyville, IL (US); Zhenkun Ma, Dallas, TX (US); Ly Phan, Malden, MA (US); Michael Rupp, Indianapolis, IN (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/136,715

(22) Filed: Apr. 30, 2002

(51) Int. Cl. A61K 31/7048; C07H 17/08

(52) U.S. Cl. 514/29; 536/7.4; 536/17.4

(57) ABSTRACT

Antibacterial compounds with improved pharmacokinetic profiles having formula (I)

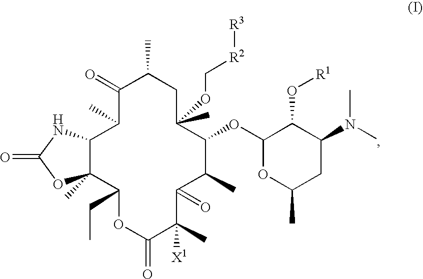

and salts, prodrugs, and salts of prodrugs thereof, processes for making the compounds and intermediates used in the processes, compositions containing the compounds, and methods for prophylaxis and treatment of bacterial infections using the compounds are disclosed.

### TECHNICAL FIELD

This invention is directed to compounds which are useful as antibacterials with improved pharmacokinetic profiles, processes for making the compounds and intermediates employed in the processes, compositions containing the compounds, and methods for prophylaxis and treatment of bacterial infections using the compounds.

### BACKGROUND OF THE INVENTION

Because the effectiveness of many drugs currently available for prophylaxis and treatment of bacterial infections is being compromised by the emergence of drug-resistant bacteria, the introduction of novel antibacterial compounds with improved pharmacokinetic profiles would be beneficial for their therapeutic value and their contribution to the antibacterial arts.

### SUMMARY OF THE INVENTION

A first embodiment of this invention, therefore, is directed to a subset of compounds, generically embraced by commonly-owned U.S. Pat. No. 6,054,435, which are useful as antibacterials with improved pharmacokinetic profiles, and salts, prodrugs, and salts of prodrugs thereof, the subset of compounds having formula (I)

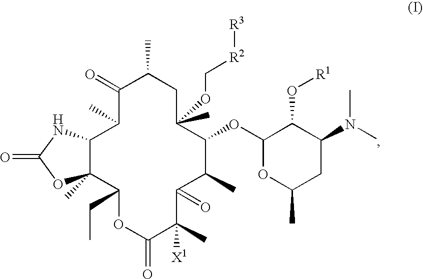

in which

R¹ is hydrogen or R^{p}, in which R^{p} is acetyl (—C(O)CH₃), benzoyl (—C(O)C₆H₅), trimethylsilyl, or triethylsilyl;

R² is —CH═CH— or —C≡C—;

R³ is tetraäzolyl or R⁴;

R⁴ is furanyl, imidazolyl, isothiazolyl, isoxazolyl, naphthyl, 1,2,3-oxadiazolyl, oxazolyl, phenyl, pyrazinyl, pyrazolyl, pyridazinyl, pyrimidinyl, pyrrolyl, 1,3,4-thiadiazolyl, thiazolyl, pyridyl(pyridinyl), thienyl(thiophenyl), 1,3,5-triazinyl, or 1,2,3-triazolyl; and

X¹ is hydrogen or fluoride,

in which each R⁴moiety is connected through a carbon atom, substituted with one tetraäzolyl substituent, and further unsubstituted or substituted with one or two substituents independently selected from the group consisting of alkyl, alkenyl, alkynyl, halo, —CN, —OH, —NH₂, —NH(alkyl), —N(alkyl)₂, —NO₂, —CF₃, —CH₂CF₃, —CF₂CF₃, —OCF₃. —OCH₂CF₃, —OCF₂CF₃, —C(O)H, —C(O)(alkyl), —C(O)OH, —C(O)O(alkyl), —C(O)NH₂, —C(O)NH(alkyl), —C(O)N(alkyl)₂, —OC(O)(alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), and —NHC(O)N(alkyl)₂, and

in which the R³ tetraäzolyl and the R⁴ tetraäzolyl substituent are connected through a carbon atom and are independently unsubstituted or substituted on a 1H or 2H nitrogen atom with a substituent selected from the group consisting of alkyl, —(CH₂)alkenyl, —(CH₂)alkynyl, and alkyl substituted with a substituent selected from the group consisting of halo, —CN, —OH, —NH₂, —NH(alkyl), —N(alkyl)₂, —CF₃, —CH₂CF₃, —CF₂CF₃, —C(O)H, ═O, —C(O)OH, —C(O)O(alkyl), —C(O)NH₂, —C(O)NH(alkyl), —C(O)N(alkyl)₂, —OC(O)(alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), and —NHC(O)N(alkyl)₂.

A second embodiment of this invention is directed to a process for making the compounds.

A third embodiment of this invention is directed to intermediates which are employed in the second embodiment.

A fourth embodiment of this invention is directed to compositions which are useful for prophylaxis or treatment of bacterial infections in a fish or a mammal, the compositions comprising a therapeutically effective amount of one or more of the compounds and an excipient.

A fifth embodiment of this invention is directed to methods for prophylaxis or treatment of bacterial infections in a fish or a mammal comprising administering to the fish or the mammal a therapeutically effective amount of one or more of the compounds.

### DETAILED DESCRIPTION OF THE INVENTION

Compounds of this invention, also referred to as “the compounds,” comprise both fixed and variable components or “moieties,” which variable moieties are identified by a capital letter and accompanying numerical or alphabetical superscript, and for which the following terms have the meanings indicated.

“Alkenyl” means monovalent, straight-chain and branched-chain hydrocarbon moieties, having two to eight carbon atoms and at least one carbon-carbon double bond, attached through a carbon atom.

Alkenyl moieties include but-1,3-dienyl, butenyl, but-2-enyl, ethenyl, 1-ethylhexen-2-yl, hex-3-enyl, 1-methylbutenyl, 2-methylbutenyl, 1-methylbut-2-enyl, 1-methylbut-1,3-dienyl, pentenyl, pent-2-enyl, pent-3-enyl, propenyl, and the like.

“Alkyl” means monovalent, saturated, straight-chain and branched-chain hydrocarbon moieties, having one to six carbon atoms, attached through a carbon atom.

Alkyl moieties include butyl, 1,1,-dimethylethyl, 1,1-dimethylpropyl, 1,2-dimethylpropyl, ethyl, 1-ethylpropyl, 2-ethylpropyl, hexyl, methyl, 2-methylpropyl, 3-methylbutyl, 1-methylpentyl, 2-methylpent-3-yl, pentyl, and the like.

“Alkynyl” means monovalent, straight-chain and branched-chain hydrocarbon moieties, having two to six carbon atoms and at least one carbon-carbon triple bond, attached through a carbon atom.

Alkynyl moieties include ethynyl (acetylenyl), pentynyl, pent-2-ynyl, pent-3-ynyl, pent-4-ynyl, 1-methylbut-2-ynyl, 2-methylbut-3-ynyl, hexynyl, hex-2-ynyl, hex-3-ynyl, hex-4-ynyl, 1-methyl-pent-2-ynyl, 1-methylenepent-3-ynyl, 1-methyl-pent-2,4-diynyl, prop-2-ynyl(propargyl), and the like.

Specific examples of R¹ moieties for the practice of this invention are hydrogen, acetyl (—C(O)CH₃), and benzoyl (—C(O)C₆H₅).

Specific examples of R² moieties for the practice of this invention are —CH═CH— and —C≡C—.

Specific examples of R³ moieties for the practice of this invention are 5-(2-allyl-2H-tetraäzol-5-yl)thien-2-yl, 5-(2-(2-cyanoethyl)-2H-tetraazol-5-yl)thien-2-yl, 5-(2-(((methoxy)carbonyl)methyl)-1H-tetraäzol-5-yl)-thien-2-yl, 2-methyl-2H-tetraäzol-5-yl, 3-(2-methyl-2H-tetraäzol-5-yl)isoxazol-5-yl, 6-(2-methyl-2H-tetraäzol-5-yl)pyridin-3-yl, 2-(2-methyl-2H-tetraäzol-5-yl)-1,3-thiazol-5-yl, 5-(2-methyl-2H-tetraäzol-5-yl)thien-2-yl, 5-(1-methyl-1H-tetraäzol-5-yl)thien-2-yl, and 5-(2H-tetraäzol-5-yl)thien-2-yl.

Specific examples of X¹ moieties for the practice of this invention are hydrogen and fluoro.

These specific moieties of the compounds may combine with the fixed moieties thereof to form a sixth embodiment of this invention, which embodiment is directed to compounds, and salts, prodrugs, and salts of prodrugs thereof, which are useful as antibacterials with improved pharmacokinetic profiles, the compounds having formula (I)

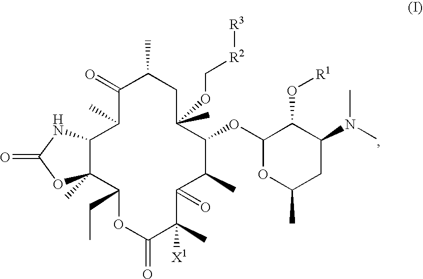

in which

R¹ is hydrogen; R² is —CH═CH— or —C≡C—; R³ is tetraäzolyl or R⁴; R⁴ is isoxazolyl, thiazolyl, pyridyl (pyridinyl), or thienyl (thiophenyl); and X¹ is hydrogen or fluoride,

in which each R⁴ moiety is substituted with one tetraäzolyl substituent, and the R³ tetraäzolyl and the R⁴ tetraäzolyl substituent are independently unsubstituted or substituted on the 1H or 2H nitrogen atom with a substituent selected from the group consisting of alkyl, —(CH₂)alkenyl, and alkyl substituted with one substituent selected from the group consisting of —CN and —C(O)O(alkyl);

a seventh embodiment of this invention, which embodiment is directed to compounds, and salts, prodrugs, and salts of thereof, having formula (I)

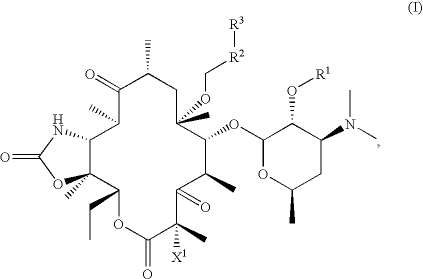

in which

R¹ is hydrogen; R² is —CH═CH— or —C≡C—; R³ is tetraäzolyl or R⁴; R⁴ is isoxazolyl, thiazolyl, pyridyl (pyridinyl), or thienyl (thiophenyl); and X¹ is hydrogen or fluoride,

in which each R⁴ moiety is substituted with one tetraäzolyl substituent, and the R³ tetrazolyl and the R⁴ tetraäzolyl substituent are independently unsubstituted or substituted at the 1H or 2H nitrogen atom with a substituent selected from the group consisting of C₁-alkyl, —(CH₂)—C₂-alkenyl, and C₁-C₂-alkyl substituted with one substituent selected from the group consisting of —CN and —C(O)O(C₁-alkyl); and

an eighth embodiment of this invention, which embodiment is directed to compounds, and salts, prodrugs, and salts of prodrugs thereof, which include

(3aS,4R,7R,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-3a,7,9,11,13,15-hexamethyl-11-(((2E)-3-(5-(2-methyl-2H-tetraazol-5-yl)thien-2-yl)prop-2-enyl)oxy)octahydro-2H-oxacyclotetradecino[4,3-d][1,3]oxazole-2,6,8,14(1H,7H,9H)-tetrone;

(3aS,4R,7R,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-3a,7,9,11,13,15-hexamethyl-11-((3-(5-(1-methyl-1H-tetraazol-5-yl)thien-2-yl)prop-2-ynyl)oxy)octahydro-2H-oxacyclotetradecino [4,3-d][1,3]oxazole-2,6,8,14(1H,7H,9H)-tetrone;

(3aS,4R,7R,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-3a,7,9,11,13,15-hexamethyl-11-(((2E)-3-(5-(2H-tetraazol-5-yl)thien-2-yl)prop-2-enyl)oxy)octahydro-2H-oxacyclotetradecino[4,3-d][1,3]oxazole-2,6,8,14(1H,7H,9H)-tetrone;

(3aS,4R,7S,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyl-11-((3-(5-(2-methyl-2H-tetraazol-5-yl)thien-2-yl)prop-2-ynyl)oxy)octahydro-2H-oxacyclotetradecino [4,3-d][1,3]oxazole-2,6,8,14(1H,7H,9H)-tetrone;

(3aS,4R,7R,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-3a,7,9,11,13,15-hexamethyl-11-((3-(5-(2-methyl-2H-tetraazol-5-yl)thien-2-yl)prop-2-ynyl)oxy)octahydro-2H-oxacyclotetradecino[4,3-d][1,3]oxazole-2,6,8,14(1H,7H,9H)-tetrone;

(3aS,4R,7S,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyl-11-(((2E)-3-(5-(2-methyl-2H-tetraazol-5-yl)thien-2-yl)prop-2-enyl)oxy)octahydro-2H-oxacyclotetradecino[4,3-d][1,3]oxazole-2,6,8,14(1H,7H,9H)-tetrone;

methyl (5-(5-(3-(((3aS,4R,7S,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyl-2,6,8,14-tetraoxotetradecahydro-2H-oxacyclotetradecino[4,3-d][1,3]oxazol-11-yl)oxy)prop-1-ynyl)thien-2-yl)-2H-tetraazol-2-yl)acetate;

(3aS,4R,7S,9R,10R,11S,13R,15R,15aR)-11-((3-(5-(2-allyl-2H-tetraazol-5-yl)thien-2-yl)prop-2-ynyl)oxy)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyloctahydro-2H-oxacyclotetradecino[4,3-d][1,3]oxazole-2,6,8,14(1H,7H,9H)-tetrone;

(3aS,4R,7S,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyl-11-((3-(6-(2-methyl-2H-tetraazol-5-yl)pyridin-3-yl)prop-2-ynyl)oxy)octahydro-2H-oxacyclotetradecino[4,3-d][1,3]oxazole-2,6,8,14(1H,7H,9H)-tetrone;

(3aS,4R,7S,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyl-11-((3-(5-(2-(2-cyanoethyl)-2H-tetraazol-5-yl)thien-2-yl)prop-2-ynyl)oxy)octahydro-2H-oxacyclotetradecino[4,3-d][1,3]oxazole-2,6,8,14(1H,7H,9H)-tetrone;

(3aS,4R,7S,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyl-11-((3-(2-methyl-2H-tetraazol-5-yl)prop-2-ynyl)oxy)octahydro-2H-oxacyclotetradecino[4,3-d][1,3]oxazole-2,6,8,14(1H,7H,9H)-tetrone;

(3aS,4R,7S,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyl-11-((3-(2-(2-methyl-2H-tetraazol-5-yl)-1,3-thiazol-5-yl)prop-2-ynyl)oxy)octahydro-2H-oxacyclotetradecino[4,3-d][1,3]oxazole-2,6,8,14(1H,7H,9H)-tetrone;

(3aS,4R,7R,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-3a,7,9,11,13,15-hexamethyl-11-((3-(2-(2-methyl-2H-tetraazol-5-yl)-1,3-thiazol-5-yl)prop-2-ynyl)oxy)octahydro-2H-oxacyclotetradecino[4,3-d][1,3]oxazole-2,6,8,14(1H,7H,9H)-tetrone;

and the like.

Compounds of this invention contain asymmetrically substituted carbon atoms in the R or S configuration, in which the terms “R” and “S” are as defined by the IUPAC 1974 Recommendations for Section E, Fundamental Stereochemistry, Pure Appl. Chem. (1976) 45, 13-10. Compounds having asymmetrically substituted carbon atoms with equal amounts of R and S configurations are racemic at those carbon atoms. Atoms with an excess of one configuration over the other are assigned the configuration which is present in the higher amount, preferably an excess of about 85%-90%, more preferably an excess of about 95%-99%, and still more preferably an excess greater than about 99%. Accordingly, this invention is meant to embrace racemic mixtures, relative and absolute stereoisomers, and mixtures of relative and absolute stereoisomers of the compounds.

Compounds of this invention may also contain carbon-carbon double bonds or carbon-nitrogen double bonds in the Z or E configuration, in which the term “Z” represents the larger two substituents on the same side of a carbon-carbon or carbon-nitrogen double bond and the term “E” represents the larger two substituents on opposite sides of a carbon-carbon or carbon-nitrogen double bond. The compounds may also exist as an equilibrium mixture of Z or E configurations.

Compounds of this invention which contain hydroxyl, amino, or carboxylic acids may have attached thereto prodrug-forming moieties. The prodrug-forming moieties are removed by metabolic processes and release the compounds having the freed hydroxyl, amino, or carboxylic acid in vivo. Prodrugs are useful for adjusting such pharmacokinetic properties of the compounds as solubility and/or hydrophobicity, absorption in the gastrointestinal tract, bioavailability, tissue penetration, and rate of clearance.

Compounds of this invention may exist as acid addition salts, basic addition salts, or zwitterions. Salts of the compounds are prepared during their isolation or following their purification. Acid addition salts of the compounds are those derived from the reaction of the compounds with an acid. For example, the acetate, adipate, alginate, bicarbonate, citrate, aspartate, benzoate, benzenesulfonate, bisulfate, butyrate, camphorate, camphorsufonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetic, trifluoroacetic, para-toluenesulfonate, undecanoate, and the like salts of the compounds and prodrugs thereof are contemplated as being embraced by this invention. When the compounds contain carboxylic acids, basic addition salts may be prepared therefrom by reaction with a base such as the hydroxide, carbonate, bicarbonate, and the like, of cations such as lithium, sodium, potassium, calcium, magnesium, and the like.

Compounds of this invention may be administered with or without an excipient. Excipients include encapsulating materials or formulation additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof, and the like. Excipients for orally administered compounds in solid dosage forms include agar, alginic acid, aluminum hydroxide, benzyl alcohol, benzyl benzoate, 1,3-butylene glycol, castor oil, cellulose, cellulose acetate, cocoa butter, corn starch, corn oil, cottonseed oil, ethanol, ethyl acetate, ethyl carbonate, ethyl cellulose, ethyl laureate, ethyl oleate, gelatin, germ oil, glucose, glycerol, groundnut oil, isopropanol, isotonic saline, lactose, magnesium hydroxide, magnesium stearate, malt, olive oil, peanut oil, potassium phosphate salts, potato starch, propylene glycol, Ringer's solution, talc, tragacanth, water, safflower oil, sesame oil, sodium carboxymethyl cellulose, sodium lauryl sulfate, sodiumphosphate salts, soybean oil, sucrose, tetrahydrofurfuryl alcohol, mixtures thereof, and the like. Excipients for ophthalmically and orally administered compounds in liquid dosage forms include benzyl alcohol, benzyl benzoate, 1,3-butylene glycol, castor oil, corn oil, cottonseed oil, ethanol, ethyl acetate, ethyl carbonate, fatty acid esters of sorbitan, germ oil, groundnut oil, glycerol, isopropanol, olive oil, polyethylene glycols, propylene glycol, sesame oil, tetrahydrofurfuryl alcohol, water, mixtures thereof, and the like. Excipients for osmotically administered compounds include chlorofluorohydrocarbons, ethanol, isopropanol, water, mixtures thereof, and the like. Excipients for parenterally administered compounds include 1,3-butanediol, castor oil, corn oil, cottonseed oil, germ oil, groundnut oil, liposomes, oleic acid, olive oil, peanut oil, Ringer's solution, safflower oil, sesame oil, soybean oil, U.S.P. or isotonic sodium chloride solution, water, mixtures thereof, and the like. Excipients for rectally and vaginally administered compounds include cocoa butter, polyethylene glycol, wax, mixtures thereof, and the like.

Compounds of this invention may be administered orally, ophthalmically, osmotically, parenterally (subcutaneously, intramuscularly, intrasternally, intravenously), rectally, topically, transdermally, and vaginally. Orally administered compounds in solid dosage forms may be administered as capsules, dragees, granules, pills, powders, tablets, and the like. Ophthalmically and orally administered compounds in liquid dosage forms may be administered as elixirs, emulsions, microemulsions, solutions, suspensions, syrups, and the like. Osmotically and topically administered compounds may be administered as creams, gels, inhalants, lotions, ointments, pastes, powders, solutions, sprays, and the like. Parenterally administered compounds may be administered as aqueous or oleaginous solutions or aqueous or oleaginous suspensions, and the like, in which suspensions comprise crystalline, amorphous, or otherwise insoluble forms of the compounds. Rectally and vaginally administered compounds may be administered as creams, gels, lotions, ointments, pastes, and the like.

Therapeutically effective amounts of compounds of this invention depend on the recepient of treatment, the disorder being treated and the severity of the disorder, the composition comprising the compounds, the time of administration, the route of administration, the duration of treatment, the potency of the compounds, and the rate of excretion of the compounds. The daily therapeutically effective amount of the compounds administered to a patient in single or divided doses range from about 0.1 to about 200 mg/kg body weight, preferably from about 0.25 to about 100 mg/kg body weight. Single dose compositions contain these amounts of the compounds or combinations of submultiples thereof.

To determine antibacterial activity of compounds of this invention, twelve petri dishes, each containing successive aqueous dilutions of test compounds in sterilized Brain Heart Infusion agar (Difco 0418-01-5) (10 mL), were inoculated with 1:100 dilutions of the representative microorganisms in TABLE 1 using a Steers replicator block (or 1:10 dilutions for slow-growing Streptococcus strains), co-incubated at 35-37° C. for 20-24 hours with a plate with a control plate having no compound, and inspected visually to provide the minimum inhibitory concentration (MIC), in μg/mL, by which is meant the lowest concentration of the test compound which yielded no growth, a slight haze, or sparsely isolated colonies on the inoculums spot as compared to growth in the control plate.

TABLE 1

| Microorganism | Code |
|---|---|
| Staphylococcus aureus NCTC10649M | AA |
| Staphylococcus aureus A5177 | BB |
| Staphylococcus aureus PIU 2043 | CC |
| Staphylococcus aureus 1775 | DD |
| Streptococcus pyrogenes EES61 | EE |
| Streptococcus pyrogenes 930 | FF |
| Streptococcus pyrogenes PIU 2548 | GG |
| Streptococcus pneumoniae ATCC 6303 | HH |
| Streptococcus pneumoniae 5979 | JJ |
| Streptococcus pneumoniae 5649 | KK |
| Enterococcus faecalis PIU 1967 | LL |
| Enterococcus faecium GYR 1632 | MM |
| Moraxella catarrhalis 2604 | NN |
| Haemophilus influenzae GYR 1435 | PP |
| Escherichia coli JUHL | QQ |

Compounds of this invention displayed antibacterial activity superior to the control, which control demonstrated no antibacterial activity. This antibacterial activity demonstrates the usefulness of the compounds as antibacterials.

It is meant to be understood that compounds of this invention having like variable moieties in combination with their fixed moieties would also be useful as antibacterials and are meant to be embraced by this invention.

It is also meant to be understood that certain metabolites of compounds of this invention, which metabolites are produced by in vitro or in vivo metabolic processes, would also be useful as antibacterials and are meant to be embraced by this invention.

It is still also meant to be understood that certain precursor compounds, which precursor compounds may be metabolized in vitro or in vivo to form compounds of this invention, are meant to be embraced by this invention.

The pharmacokinetic profiles of EXAMPLE 20, EXAMPLE 22, EXAMPLE 29 and EXAMPLE 31 were evaluated using cassette dosing protocols in dog. The cassette dosing protocol in dogs combined three test compounds with the reference compound (EXAMPLE 104 of commonly-owned U.S. Pat. No. 5,866,549), each at a dose of 1 mg/kg. The compounds for each cassette were formulated as a solution in an aqueous vehicle containing 10% ethanol and one equivalent hydrochloric acid. Groups of three male beagle dogs received either an intravenous dose (IV) or an oral dose (PO). Sequential blood samples were taken from each animal for 24 hours after dosing. The plasma concentrations of the test compound and reference compounds were simultaneously determined using HPLC-MS/MS following liquid-liquid extraction of the samples. The results are shown in TABLE 2, in which ClogP is plasma clearance; T_{1/2} and Tₘₐₓ are expressed in hours; Cₘₐₓ is expressed in μg/mL; AUC is expressed in μg·h/mL; F is percent bioavaibility; and volumes of distibution Vc and V_{β} are volume of the central compartment and terminal phase distribution, respectively.

TABLE 2

| | | T_{½} | V_{c} | V_{β} | AUC | CLp | T_{½} | Cmax | Tmax | AUC | F |
|---|---|---|---|---|---|---|---|---|---|---|---|
| EXAMPLE | Dose | (IV) | (IV) | (IV) | (IV) | (IV) | (PO) | (PO) | (PO) | (PO) | (PO) |
| Standard | 1 | 4.7 | 1.8 | 2.4 | 3.01 | 0.35 | 4.2 | 0.21 | 1.1 | 1.79 | 59.00 |
| 3B | 1 | 14.4 | 1.6 | 3.1 | 7.29 | 0.16 | 11.1 | 0.25 | 2 | 5.14 | 73.60 |
| 4B | 1 | 8.4 | 0.9 | 1.1 | 11.52 | 0.09 | 7.8 | 0.49 | 1.5 | 6.77 | 63.00 |
| 7B | 1 | 9 | 0.9 | 1.2 | 11.9 | 0.09 | 6.7 | 0.72 | 1.3 | 9.66 | 80.30 |
| 8B | 1 | 22.9 | 1.1 | 2.3 | 16.13 | 0.07 | 17.2 | 0.48 | 4.5 | 12.6 | 81.10 |

The data in TABLE 2 show pharmacokinetic profiles of the compounds are characterized by low plasma clearance values, long half lives, and high oral bioavailabilities.

Compounds of this invention may also be prepared by synthetic chemical processes, examples of which synthetic chemical processes, and intermediates employed in the processes, are shown hereinbelow. It is meant to be understood that the order of the steps in the processes may be varied, like reagents, solvents, and reaction conditions may be substituted for those specifically mentioned, and vulnerable moieties may be protected and deprotected during the process.

Abbreviations used are THF for tetrahydrofuran, DME for 1,2-dimethoxyethane; and DMF for N,N-dimethylformamide.

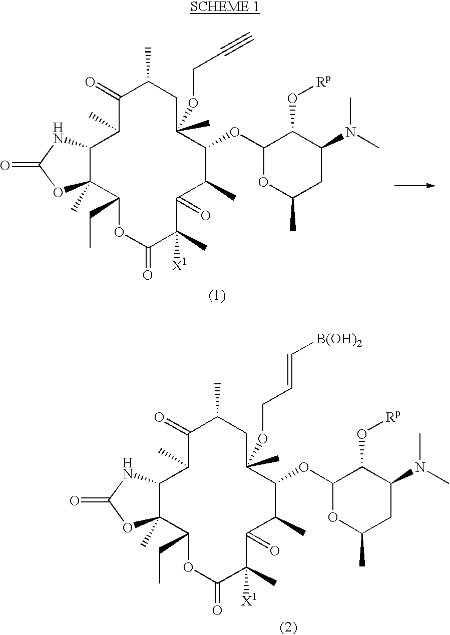

The compound having formula (1), in which X¹ is hydrogen, may be prepared as described in commonly-owned U.S. Pat. No. 5,866,549.

The compound having formula (1), in which X¹ is fluoro, may be prepared as described in commonly-owned U.S. Pat. No. 6,124,269.

Compounds having formula (1) may be converted to compounds having formula (2) by (a) reacting the former, a borane, and optionally, a first additive; and (b) reacting the product of step (a) and water. Boranes include borane.tetrahydrofuran, borane.dimethylsulfide, borene.1,4-oxathiane, and the like. Additives include 2-methyl-2-butene and the like. Step (a) is typically conducted from about −5° C. to about 5° C., for about 1 to about 10 hours, in solvents such as diethyl ether, dioxane, DME, THF, and the like. Step (b) is typically conducted, without isolation of the product of Step (a), from about −5° C. to about 5° C., for about 1 to about 10 hours, in mixtures of water and benzene, diethyl ether, dioxane, DME, THF, toluene, and the like.

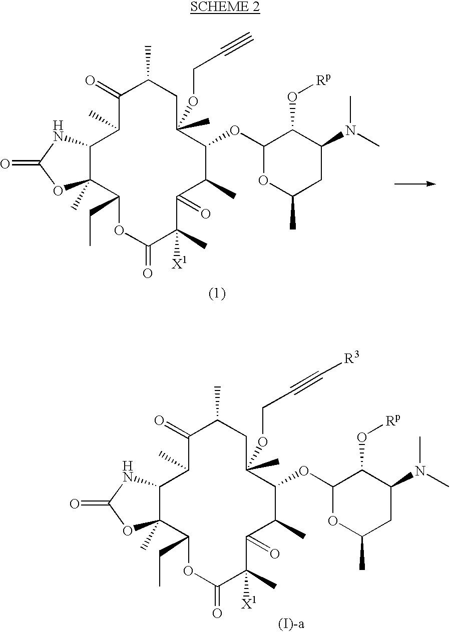

Compounds having formula (1) may be converted to compounds having formula (I)-a by reacting the former, compounds having formula (3),

X²—R³ (3),

a first base, a coupling catalyst, copper(I) iodide, and, optionally, a second additive. First bases include N,N-diisopropylethylamine, triethylamine, and the like.

Coupling catalysts include

dichlorobis(triphenylphosphine)palladium(II),

tris(dibenzylideneacetone)dipalladium(0),

tetrakis(triphenylphosphine)palladium(0),

dichlorobis(triphenylphosphine)nickel(II), and the like.

Second additives include 1,2-bis(diphenylphosphino)ethane, 1,3-bis(diphenylphosphino)propane, triphenylphosphine, triphenylarsine, mixtures thereof, and the like. The reaction is typically conducted from about 50° C. to about 80° C., for about 12 to about 48 hours, in solvents such as acetonitrile, benzene, diethyl ether, DME, dioxane, THF, toluene, and the like.

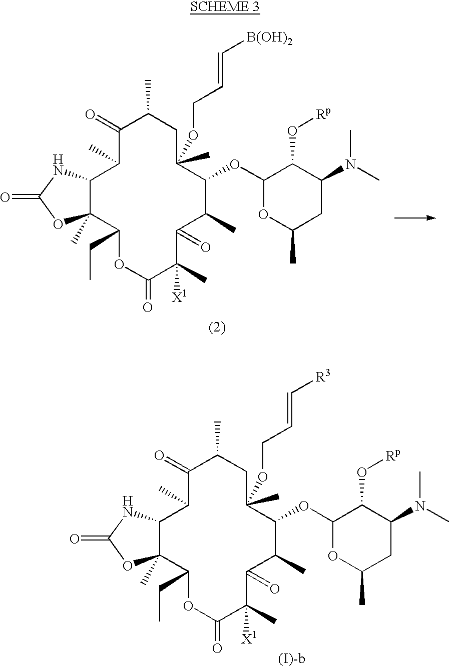

Compounds having formula (2) may be converted to compounds having formula (I)-b by reacting the former, the compounds having formula (3), the coupling catalyst, a second base, and, optionally, the second additive.

Second bases include sodium carbonate, potassium carbonate, cesium carbonate, cesium fluoride, N,N-diisopropylethylamine, and triethylamine.

The reaction is typically conducted from about 50° C. to about 80° C., for about 12 to about 48 hours, in solvents such as acetonitrile, benzene, diethyl ether, DME, dioxane, THF, toluene, and the like.

Other processes for making compounds of this invention are well-known in the art.

The compounds and processes of this invention will be better understood in connection with the following examples.

### EXAMPLE 1

This example was prepared according to EXAMPLE 246 in commonly-owned U.S. Pat. No. 5,866,549 and substituting 90% technical grade benzoic anhydride for acetic anhydride in Step 246d.

### EXAMPLE 2

This example was prepared according to EXAMPLE 4 in commonly-owned U.S. Pat. No. 6,124,269 and substituting EXAMPLE 1 for the “compound 14 of Scheme 4.”

### EXAMPLE 3

A solution of 1M borane.THF (119 mL) at 0° C. was treated with a solution of 2-methyl-2-butene (25.1 mL) in THF (75 mL) at 0° C., stirred for 45 minutes, treated with a solution of EXAMPLE 2 (15 g) in THF (100 mL) at 0° C., stirred for 2 hours, treated with 10% K₂CO₃, and extracted with ethyl acetete; and the extract was dried (Na₂SO₄), filtered, and concentrated. A solution of the concentrate in ethyl acetate (50 mL) was treated with hexane (500 mL) and filtered; and the filtrant was flash chromatographed on silica gel with 50-70% acetone/hexanes.

### EXAMPLE 4

This example was prepared by substituting EXAMPLE 1 for EXAMPLE 2 in EXAMPLE 3.

### EXAMPLE 5

This example was prepared by substituting EXAMPLE 2 for “the compound from Step 1a” in Step 1b of EXAMPLE 1 of commonly-owned U.S. Pat. No. 6,124,269.

### EXAMPLE 6 AND EXAMPLE 7

5-(5-bromothien-2-yl)-2-methyl-2H-tetraäzole and 5-(5-bromothien-2-yl)-1-methyl-1H-tetraäzole

A solution of 5-(5-bromothien-2-yl)-1H (and 2H)-tetraäzole (750 mg) and diisopropylethylamine (850 μL) in acetonitrile (25 mL) and methanol (2.5 mL) at 0° C. was treated with 2M (trimethylsilyl)diazomethane in THF (2.4 mL), stirred for 3 hours at 25° C., treated with ethyl acetate, washed with water and brine, and dried (Na₂SO₄), filtered, and concentrated; and the concentrate was flash chromatographed on silica gel with 99:1 dichloromethane/methanol.

### EXAMPLE 8

5-(5-bromothien-2-yl)-2-((2-trimethylsilyl)ethoxy)methyl-2H-tetraäzole

A solution of 5-(5-bromothien-2-yl)-2H-tetraäzole and 5-(5-bromothien-2-yl)-1H-tetraäzole (100 mg), triethylamine (120 μL), and 2-(trimethylsilyl)ethoxymethylchloride (80 μL) in THF at 25° C. was stirred for 2 hours, treated with ethyl acetate, washed with water and brine, and dried (Na₂SO₄), filtered, and concentrated; and the concentrate was flash chromatographed on silica gel with dichloromethane.

### EXAMPLE 2E

A solution of 5-(5-bromothien-2-yl)-1H-tetraäzole and 5-(5-bromothien-2-yl)-2H-tetraäzole (300 mg) in ethanol (5 mL) was treated with silver nitrate in water (1 mL), stirred for 30 minutes, treated with concentrated ammonium hydroxide (5 mL), and filtered. A slurry of the filtrant in chloroform (10 mL) was treated with allyl bromide (280 μL), stirred at 65° C. for 18 hours, and filtered; and the filtrant was flash chromatographed on silica gel with dichloromethane.

### EXAMPLE 10

A solution of 5-(5-bromothien-2-yl)-1H-tetraäzole and 5-(5-bromothien-2-yl)-2H-tetraäzole (500 mg), acrylonitrile (210 μL), and triethylamine (900 μL) in isopropanol-(2 mL) was stirred at 110° C. in a sealed tube for 18 hours and concentrated; and the concentrate was flash chromatographed on silica gel with 99:1 dichloromethane/methanol.

### EXAMPLE 11

This example was prepared by substituting ethyl bromoacetate for allyl bromide in EXAMPLE 9.

### EXAMPLE 12

A mixture of sodium hydroxide (14.2 g) and 1H-tetraäzole and 2H-tetraäzole (25 g) in water (600 mL) at 25° C. was stirred until homogeneous, treated sequentially with dichloromethane (600 mL), dimethyl sulfate (47.2 g), and n-tetrabutylammonium bromide (5.7 g), stirred for 14 hours, separated from the water layer, and distilled at 1 atm with collection of the 143° C. fraction.

### EXAMPLE 13

A solution of EXAMPLE 12 (3.5 g) in THF (150 mL) at −78° C. was treated with 2.5 M n-butyllithium in cyclohexane (20.8 mL) over 35 minutes, stirred for 15 minutes, treated with a solution of N-iodosuccinimide (10.3 g) in THF (75 mL) over 20 minutes, stirred for 30 minutes, warmed to 0° C. and stirred for 40 minutes, warmed to 25° C. and stirred for 10 minutes, treated with diethyl ether, washed with 5% KH₂PO₄, water, and brine, and dried (Na₂SO₄), filtered, and concentrated; and the concentrate was flash chromatographed on silica gel with 1:1 dichloromethane/hexanes.

### EXAMPLE 14

A solution of EXAMPLE 13 (1.78 g) in toluene (19 mL) was treated with 2-tributylstannylthiazole (3.65 g) and tetrakis(triphenylphosphine)palladium(0) (196 mg), stirred at 95° C. for 16 hours, and concentrated; and the concentrate was flash chromatographed on silica gel with dichloromethane then 25:75 acetone/hexanes.

### EXAMPLE 15

A solution of 2.5M n-butyllithium in hexanes at −78° C. (1.8 mL) in diethyl ether (16 mL) was treated with a solution of EXAMPLE 14 (600 mg) in tetrahydrofuran (18 mL) over 1 hour, stirred for 45 minutes, treated with 1M trimethylstannyl chloride in THF (4.5 mL) in tetrahydrofuran (4 mL) over 10 minutes, stirred for 1 hour, and concentrated; and the concentrate was treated with ethyl ether (50 mL), filtered through diatomaceous earth (Celite®), and concentrated.

### EXAMPLE 16

A solution of the EXAMPLE 15 concentrate in THF (35 mL) at 25° C. was treated with iodine (1 g) in THF (15 mL) over 5 minutes, stirred for 2 hours, treated with ethyl acetate, washed with 5% Na₂CO₃, 10% Na₂S₂O₃, and brine, and dried (Na₂SO₄), filtered, and concentrated; and the concentrate was flash chromatographed on silica gel with of 1:1 hexanes/dichloromethane then dichloromethane.

### EXAMPLE 17

A solution of 5-bromo-2-cyanopyridine (1 g), ammonium chloride (4.3 g), and sodium azide (5.32 g) in DMF (50 mL) was stirred at 130° C. for 4 hours, cooled to ambient temperature, diluted with dichloromethane, washed with water and brine, and dried (Na₂SO₄), filtered and concentrated.

### EXAMPLE 18

A mixture of EXAMPLE 18 (582 mg) and diisopropylethylamine (650 mL) in acetonitrile (10 mL) and methanol (2 mL) at 0° C. was treated with trimethylsilyldiazomethane (1.93 mL) over 3 minutes, stirred for 1.5 hours, diluted with ethyl acetate, washed with 5% NaHCO₃, water, and brine, and dried (Na₂SO₄), filtered and concentrated; and the concentrate was flash column chromatographed on silica gel with 4:1 hexanes/ethyl acetate.

### EXAMPLE 19

A solution of EXAMPLE 2 (1.4 g), EXAMPLE 6 (500 mg) triethylamine (2 mL), and 1,2-bis(diphenylphosphino)ethane (73.7 mg) in acetonitrile (10 mL) at 25° C. was treated with tris(dibenzylideneacetone)dipalladium(0) (84.7 mg) and copper(I)iodide (17.6 mg), stirred for 10 minutes at 25° C. and 85° C. for 24 hours, and concentrated; and the concentrate was flash chromatographed on silica gel with 20-33% acetone/hexanes.

### EXAMPLE 20

A solution of EXAMPLE 19 (1.36 g) in methanol (500 mL) at reflux was stirred for 8 hours and concentrated; and the concentrate was flash chromatographed on silica gel with 2-4% methanol/dichloromethane.

### EXAMPLE 21

A mixture of EXAMPLE 4 (3 g), EXAMPLE 6 (1.03 g), and sodium carbonate (1.21 g) in toluene (50 mL) and water (25 mL) was treated with tetrakis(triphenylphosphine)-palladium(0) (440 mg), stirred at reflux for 16 hours, and concentrated; and the concentrate was flash chromatographed on silica gel with 97.9:2:0.1 dichloromethane/methanol/concentrated ammonium hydroxide.

### EXAMPLE 22

A solution of EXAMPLE 21 (1.03 g) in methanol (15 mL) was stirred at reflux for 8 hours and concentrated; and the concentrate was flash chromatographed on silica gel with 94.9:5:0.1 dichloromethane/methanol/concentrated ammonium hydroxide.

### EXAMPLE 23

A mixture of EXAMPLE 4 (670 mg), EXAMPLE 7 (230 mg), and sodium carbonate (270 mg) in toluene (12 mL) and water (6 mL) was treated with tetrakis(triphenylphosphine)palladium(0) (110 mg), stirred at reflux for 16 hours, and concentrated; and the concentrate was flash chromatographed on silica gel with 97.9:2:0.1 dichloromethane/methanol/concentrated ammonium hydroxide.

### EXAMPLE 24

This example was prepared by substituting EXAMPLE 23 for EXAMPLE 19 in EXAMPLE 20.

### EXAMPLE 25

A mixture of EXAMPLE 4 (1.4 g), EXAMPLE 8 (710 mg), and sodium carbonate (570 mg) in toluene (25 mL) and water (12 mL) was treated with tetrakis(triphenylphosphine)-palladium(0) (200 mg), stirred at reflux for 16 hours, and concentrated; and the concentrate was flash chromatographed on silica gel with 30% to 50% to 70% acetone/hexanes.

### EXAMPLE 26

A solution of EXAMPLE 25 (600 mg) in methanol (15 mL) was stirred at reflux for 6 hours and concentrated; and the concentrate was flash chromatographed on silica gel with 94.9:5:0.1 dichloromethane/methanol/concentrated ammonium hydroxide.

### EXAMPLE 27

A mixture of EXAMPLE 26 (320 mg), 4 Å molecular sieves, and 1M tetrabutylammonium fluoride in THF (1.751 mL) in THF (15 mL) was stirred at reflux for 4 hours and cooled, treated with chloroform, filtered, washed with water, and dried (Na₂SO₄), filtered, and concentrated; and the concentrate was flash chromatographed on silica gel with 94.5:5:0.5 dichloromethane/methanol/concentrated ammonium hydroxide.

### EXAMPLE 28

A solution of EXAMPLE 2 (1 g), EXAMPLE 6 (430 mg) triethylamine (1.4 mL), and 1,2-bis(diphenylphosphino)ethane (54 mg) in acetonitrile (7 mL) at 25° C. was treated with tris(dibenzylideneacetone)dipalladium(0) (62 mg) and copper(I) iodide (12.8 mg), stirred for 1 hour at 25° C. and at 85° C. for 24 hours, and concentrated; and the concentrate was flash chromatographed on silica gel with 20-33% acetone/hexanes.

### EXAMPLE 29

A solution of EXAMPLE 28 (790 mg) in methanol (15 mL) was stirred at reflux for 16 hours and concentrated; and the concentrate was flash chromatographed on silica gel with 97:2:1 dichloromethane/methanol/concentrated ammonium hydroxide.

### EXAMPLE 30

A solution of EXAMPLE 3 (600 mg), EXAMPLE 6 (365 mg), and potassium carbonate (206 mg) in acetone (5 mL) and water (5 mL) was treated with palladium(II) acetate (8.4 mg), stirred at 65° C. for 16 hours and cooled, treated with ethyl acetate, washed with water and brine, and dried (Na₂SO₄), filtered, and concentrated.

### EXAMPLE 31

A solution of EXAMPLE 30 (600 mg) in methanol (15 mL) was stirred at reflux for 16 hours and concentrated; and the concentrate was flash chromatographed on silica gel with 98:2 dichloromethane/methanol.

### EXAMPLE 32

A solution of EXAMPLE 2 (500 mg), EXAMPLE 11 (250 mg) triethylamine (3 mL), and 1,2-bis(diphenylphosphino)ethane (26 mg) in acetonitrile (10 mL) at 25° C. was treated with tris(dibenzylideneacetone)dipalladium(0) (30 mg) and copper(I) iodide (2.5 mg), stirred for 10 minutes at 25° C. and 85° C. for 24 hours, and concentrated; and the concentrate was flash chromatographed on silica gel with 20-33% acetone/hexanes.

### EXAMPLE 33

This example was prepared by substituting EXAMPLE 28 for EXAMPLE 19 in EXAMPLE 20.

### EXAMPLE 34

This example was prepared by substituting EXAMPLE 9 for EXAMPLE 6 in EXAMPLE 19.

### EXAMPLE 35

A solution of EXAMPLE 30 (50 mg) in methanol (5 mL) was stirred at reflux for 16 hours and concentrated; and the concentrate was flash chromatographed on silica gel with 97:2:1 dichloromethane/methanol/concentrated ammonium hydroxide.

### EXAMPLE 36

A solution of EXAMPLE 5 (417 mg), EXAMPLE 18 (153 mg), and triethylamine (1 mL) in acetonitrile (10 mL) at 25° C. was treated with bis(triphenylphosphine)palladium(II)acetate (44.7 mg) and copper(I) iodide (190.4 mg), stirred at 90° C. for 4 hours, and concentrated; and the concentrate was flash chromatographed on silica gel with 2% methanol/dichloromethane.

### EXAMPLE 37

A solution of EXAMPLE 5 (630 mg), EXAMPLE 10 (300 mg) triethylamine (3 mL), and 1,2-bis(diphenylphosphino)ethane (38 mg) in acetonitrile (3 mL) at 25° C. was treated with tris(dibenzylideneacetone)dipalladium(0) (40 mg) and copper(I) iodide (4 mg), stirred for 18 hours at 85° C., and concentrated; and the concentrate was flash chromatographed on silica gel with 94.5:5:0.1 dichloromethane/methanol/concentrated ammonium hydroxide.

### EXAMPLE 38

A solution of EXAMPLE 2 (700 mg), EXAMPLE 13 (230 mg), and triethylamine (5 mL) in acetonitrile (10 mL) at 25° C. was treated with bis(triphenylphosphine)palladium(II) acetate (32 mg) and copper(I) iodide (4 mg), stirred at 90° C. for 4 hours, and concentrated; and the concentrate was flash chromatographed on silica gel with 1:1 hexane/acetone.

### EXAMPLE 39

This example was prepared by substituting EXAMPLE 38 for EXAMPLE 19 in EXAMPLE 20.

### EXAMPLE 40

A solution of EXAMPLE 2 (800 mg) and triethylamine (3 ml) in acetonitrile (16 mL) at 25° C. was treated sequentially with EXAMPLE 16 (324 mg), dichlorobis(triphenylphosphine)palladium(II) (52 mg), and copper(I)iodode (4 mg), stirred at 65° C. for 16 hours and concentrated; and the concentrate was flash chromatographed on silica gel with 25:75 acetone/hexanes.

### EXAMPLE 41

This example was prepared by substituting EXAMPLE 40 for EXAMPLE 19 in EXAMPLE 20.

### EXAMPLE 42

A solution of EXAMPLE 1 (220 mg) and triethylamine (750 μl) in acetonitrile (4 mL) at 25° C. was treated sequentially with EXAMPLE 16 (100 mg), dichlorobis(triphenylphosphine)palladium(II) (15 mg), and copper(I)iodode (1 mg), stirred at 65° C. for 16 hours and concentrated; and the concentrate was flash chromatographed on silica gel with 25:75 acetone/hexanes.

### EXAMPLE 43

A solution of EXAMPLE 42 (190 mg) in methanol (8 mL) was stirred reflux for 16 hours and concentrated; and the concentrate was flash chromatographed on silica gel with 99:1 dichloromethane/methanol then 98.5:1:0.5 dichloromethane/methanol/concentrated ammonium hydroxide.

### EXAMPLE 20

¹³C NMR (75 MHz, CDCl₃) δ216.6, 204.0, 203.8, 165.9, 165.7 (C-1) ,160.7, 157.2, 133.0, 129.9, 127.5, 125.3, 104.1, 91.9, 83.4, 80.2, 80.0, 78.7, 78.5, 70.3, 69.7, 65.8, 58.1, 51.1, 44.1, 40.5, 40.2, 39.4, 38.4, 37.4, 28.1, 25.3, 25.1, 22.2, 21.1, 20.2, 17.6, 15.3, 13.7, 13.2, 10.6.

### EXAMPLE 22

¹³C NMR (75 MHz, CDCl₃) δ217.1, 205.23, 169.6, 161.2, 157.5, 144.0, 128.1, 126.9, 126.6, 126.1, 102.9, 83.4, 78.6, 77.6, 76.4, 70.2, 69.5, 65.9, 63.8, 58.2, 50.9, 46.2, 45.0, 40.2, 39.3, 38.9, 37.3, 28.3, 22.7, 21.2, 20.3, 18.1, 14.4, 14.1, 13.7, 10.7.

### EXAMPLE 24

¹³C NMR (75 MHz, CDCl₃) δ217.4, 205.1, 169.5, 157.5, 149.8, 146.5, 130.76, 128.4, 126.7, 125.63, 122.4, 102.9, 83.52, 78.63, 77.4, 76.3, 70.1, 69.5, 65.8, 63.5, 58.1, 50.8, 46.3, 44.9, 40.2, 38.8, 37.2, 35.1, 28.2, 22.6, 21.2, 20.4, 17.9, 14.34, 14.31, 13.58, 13.55, 10.7.

### EXAMPLE 27

¹³C NMR (75 MHz, CDCl₃) δ217.2, 205.2, 169.6, 157.6, 156.7, 141.5, 126.5, 126.3, 126.1, 125.7, 102.6, 83.6, 78.6, 77.2, 70.1, 69.1, 65.9, 63.9, 58.6, 52.2, 50.8, 45.0, 40.2, 38.8, 37.3, 30.9, 25.5, 23.7, 22.6, 21.1, 20.2, 19.6, 18.1, 14.5, 14.2, 13.6, 10.6.

### EXAMPLE 29

¹³C NMR (75 MHz, CDCl₃,) δ216.9, 205.2, 169.4, 157.7, 133.2, 130.0, 127.5, 125.4, 103.1, 91.4, 83.6, 79.5, 78.7, 77.4, 77.2, 70.3, 69.6, 65.9, 58.2, 51.7, 51.1, 46.7, 44.8, 40.2, 39.5, 38.8, 37.4, 28.2, 22.5, 21.2, 19.7, 18.0, 14.7, 14.5, 13.6, 13.5, 10.6.

### EXAMPLE 31

¹³C NMR (CDCl₃, 75 MHz) δ217.6, 204.0, 203.6, 166.0, 165.6, 161.3, 157.0, 143.9, 128.3, 127.8, 127.7, 126.7, 126.2, 103.9, 99.2, 83.3, 79.9, 79.2, 79.0, 70.3, 69.6, 65.9, 63.7, 58.2, 44.2, 40.6, 40.2, 39.4, 38.8, 37.5, 28.4, 25.5, 25.3, 25.2, 22.4, 21.1, 20.9, 17.7, 15.5, 13.8, 13.3, 10.8.

### EXAMPLE 35

¹³C NMR (CDCl3, 75 MHz) δ216.5, 204.1, 203.7, 165.9, 165.6, 160.8, 157.2, 132.9, 129.9, 129.6, 127.6, 125.3, 120.9, 104.1, 98.9, 96.2, 91.9, 83.4, 80.2, 80.1, 78.7, 78.6, 77.0, 70.3, 69.6, 65.8, 58.1, 55.4, 51.1, 44.1, 40.5, 40.2, 38.4, 37.4, 28.2, 25.3, 25.0, 22.2, 21.1, 20.2, 17.6, 15.3, 13.7, 13.2, 10.6.

### EXAMPLE 36

¹³C NMR (75 MHz, CDCl₃) δ216.9, 204.4, 203.8, 166.1, 165.8, 157.3, 151.9, 151.8, 143.3, 139.9, 123.7, 121.8, 103.8, 99.0, 96.3, 92.2, 83.6, 81.7, 80.4, 79.7, 78.6, 70.1, 69.3, 65.9, 57.9, 50.9, 46.1, 44.2, 40.6, 40.3, 38.3, 37.4, 37.0, 25.3, 25.0, 22.2, 21.0, 20.3, 17.6, 15.3, 13.6, 13.2, 10.6.

### EXAMPLE 37

¹³C NMR (75 MHz, CDCl₃) δ216.62, 204.1, 203.7, 165.9, 165.6, 161.2, 157.2, 133.0, 130.7, 129.2, 128.1, 125.84, 115.3, 104.1, 98.9, 96.2, 92.2, 83.5, 80.2, 79.9, 78.6, 78.5, 78.4, 73.9, 70.3, 69.7, 65.8, 58.0, 51.1, 48.1, 44.1, 40.5, 40.2, 38.4, 37.4, 30.8, 28.1, 25.4, 25.0, 22.2, 21.1, 20.2, 18.1, 17.6, 15.3, 13.7, 13.2, 10.6.

### EXAMPLE 39

¹³C NMR (CDCl3, 75 MHz) δ216.0, 204.2, 203.8, 165.9, 165.6, 157.4, 150.6, 104.2, 98.8, 91.3, 83.2, 80.6, 79.1, 72.9, 70.2, 69.7, 65.7, 58.3, 50.4, 44.0, 40.2, 39.7, 38.0, 37.6, 30.8, 28.1, 25.5, 25.4, 22.3, 21.1, 20.3, 17.7, 15.5, 13.9, 13.5, 10.6.

### EXAMPLE 41

¹³C NMR (CDCl₃, 75 MHz) δ216.9, 204.2, 203.8, 166.0, 165.8, 160.4, 157.3, 154.4, 148.4, 104.2, 95.2, 83.5, 80.4, 79.9, 78.6, 75.1, 70.3, 69.8, 65.8, 58.1, 51.2, 44.2, 40.6, 40.2, 39.8, 38.5, 37.5, 28.1, 25.3, 25.0, 22.2, 21.2, 20.2, 17.6, 15.3, 13.7, 13.2, 10.6.

### EXAMPLE 43

¹³C NMR (CDCl₃, 75 MHz) δ217.1, 205.2, 169.6, 160.4, 157.8, 154.3, 148.6, 121.2, 103.1, 94.7, 83.5, 79.8, 77.3, 77.2, 75.2, 70.2, 58.1, 51.6, 51.0, 46.7, 44.8, 40.2, 39.8, 38.8, 37.4, 28.2, 22.4, 21.2, 19.6, 17.9, 14.7, 14.5, 13.6, 13.5, 10.5.

The foregoing is merely illustrative of the invention and is not intended to limit the same to the disclosed compounds and proceses. Variations and changes which are obvious to one skilled in the art are intended to be within the scope and nature of the invention as defined in the claims.

What is claimed is:

1. A compound, or a salt, prodrug, or salt of a prodrug thereof, having formula (I) 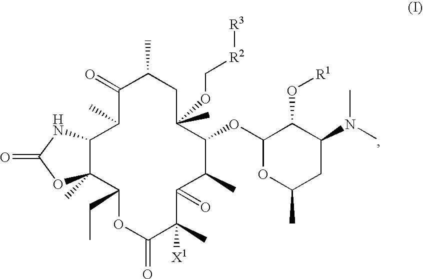
in which
R¹ is hydrogen or R^{p}, in which R^{p} is acetyl (—C(O)CH₃), benzoyl(—C(O)C₆H₅), trimethylsilyl, or triethylsilyl;
R² is —CH═CH— or —C≡C—;
R³ is tetraäzolyl or R⁴;
R⁴ is furanyl, imidazolyl, isothiazolyl, isoxazolyl, naphthyl, 1,2,3-oxadiazolyl, oxazolyl, phenyl, pyrazinyl, pyrazolyl, pyridazinyl, pyrimidinyl, pyrrolyl, 1,3,4-thiadiazolyl, thiazolyl, pyridyl (pyridinyl), thienyl (thiophenyl), 1,3,5-triazinyl, or 1,2,3-triazolyl; and
X¹ is hydrogen or fluoride,
in which each R⁴ moiety is connected through a carbon atom, substituted with one tetraäzolyl substituent, and further unsubstituted or substituted with one or two substituents independently selected from the group consisting of alkyl, alkenyl, alkynyl, halo, —CN, —OH, —NH₂, —NH(alkyl), —N(alkyl)₂, —NO₂, —CF₃, —CH₂CF₃, —CF₂CF₃, —OCF₃, —OCH₂CF₃, —OCF₂CF₃, —C(O)H, —C(O)(alkyl), —C(O)OH, —C(O)O(alkyl), —C(O)NH₂, —C(O)NH(alkyl), —C(O)N(alkyl)₂, —OC(O)(alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), and —NHC(O)N(alkyl)₂, and
in which the R³ tetraäzolyl and the R⁴ tetraäzolyl substituent are connected through a carbon atom and are independently unsubstituted or substituted on a 1H or 2H nitrogen atom with a substituent selected from the group consisting of alkyl, —(CH₂)alkenyl, —(CH₂)alkynyl, and alkyl substituted with a substituent selected from the group consisting of halo, —CN, —OH, —NH₂, —NH(alkyl), —N(alkyl)₂, —CF₃, —CH₂CF₃, —CF₂CF₃, —C(O)H, ═O, —C(O)OH, —C(O)O(alkyl), —C(O)NH₂, —C(O)NH(alkyl), —C(O)N(alkyl)₂, —OC(O)(alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), and —NHC(O)N(alkyl)₂.

2. A compound of claim 1, or a salt, prodrug, or salt of a prodrug thereof, having formula (I) 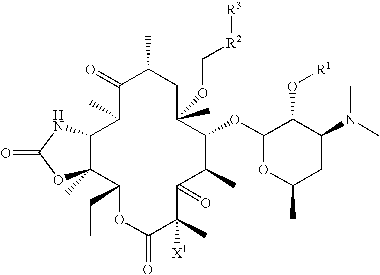
in which
R¹ is hydrogen; R² is —CH═CH— or —C≡C—; R³ is tetraäzolyl or R⁴; R⁴ is isoxazolyl, thiazolyl, pyridyl, or thienyl; and X¹ is hydrogen or fluoride,
in which each R⁴ moiety is substituted with one tetraäzolyl substituent, and the R³ tetraäzolyl and the R⁴ tetraäzolyl substituent are independently unsubstituted or substituted at the 1H or 2H nitrogen atom with a substituent selected from the group consisting of alkyl, —(CH₂)alkenyl, and alkyl substituted with one substituent selected from the group consisting of —CN and —C(O)O(alkyl).

3. A compound of claim 1, or a salt, prodrug, or salt of a prodrug thereof, having formula (I) 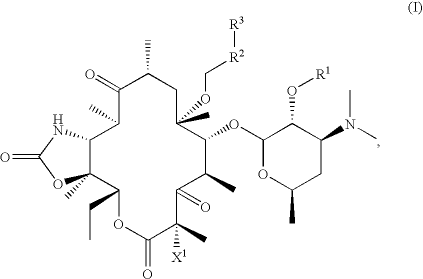
in which
R¹ is hydrogen; R² is —CH═CH— or —C≡C—; R³ is tetraäzolyl or R⁴; R⁴ is or isoxazolyl, thiazolyl, pyridyl, and thienyl; and X¹ is hydrogen or fluoride,
in which each R⁴ moiety is substituted with one tetraäzolyl substituent, and the R³ tetraäzolyl and the R⁴ tetraäzolyl substituent are independently unsubstituted or substituted at the 1H or 2H nitrogen atom with a substituent selected from the group consisting of C₁-alkyl, —(CH₂)—C₂-alkenyl, and C₁-C₂-alkyl substituted with one substituent selected from the group consisting of —CN and —C(O)O(C₁-alkyl).

4. A composition for prophylaxis or treatment of bacterial infections in a fish or a mammal, the compositions comprising a therapeutically effective amount of a compound of claim 1 and an excipient.

5. A method for prophylaxis or treatment of bacterial infections in a fish or a mammal comprising administering to the fish of the mammal a therapeutically effective amount of a compound of claim 1.

6. A compound of claim 1, or a salt, prodrug, or salt of a prodrug thereof, which is
(3aS,4R,7R,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyl-11-(((2E)-3-(5-(2-methyl-2H-tetraazol-5-yl)thien-2-yl)prop-2-enyl)oxy)octahydro-2H-oxacyclotetradecino[4,3-d][1,3]oxazole-2,6,8,14(1H,7H,9H)-tetrone;
(3aS,4R,7R,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-3a,7,9,11,13,15-hexamethyl-11-((3-(5-(1-methyl-1H-tetraazol-5-yl)thien-2-yl)prop-2-ynyl)oxy)octahydro-2H-oxacyclotetradecino[4,3-d][1,3]oxazole-2,6,8,14(1H,7H,9H)-tetrone;
(3aS,4R,7R,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-3a,7,9,11,13,15-hexamethyl-11-(((2E)-3-(5-(2H-tetraazol-5-yl)thien-2-yl)prop-2-enyl)oxy)octahydro-2H-oxacyclotetradecino[4,3-d][1,3]oxazole-2,6,8,14(1H,7H,9H)-tetrone;
(3aS,4R,7S,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyl-11-((3-(5-(2-methyl-2H-tetraazol-5-yl)thien-2-yl)prop-2-ynyl)oxy)octahydro-2H-oxacyclotetradecino[4,3-d][1,3]oxazole-2,6,8,14(1H,7H,9H)-tetrone;
(3aS,4R,7R,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-3a,7,9,11,13,15-hexamethyl-11-((3-(5-(2-methyl-2H-tetraazol-5-yl)thien-2-yl)prop-2-ynyl)oxy)octahydro-2H-oxacyclotetradecino[4,3-d][1,3]oxazole-2,6,8,14(1H,7H,9H)-tetrone;
(3aS,4R,7S,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyl-11-(((2E)-3-(5-(2-methyl-2H-tetraazol-5-yl)thien-2-yl)prop-2-enyl)oxy)octahydro-2H-oxacyclotetradecino[4,3-d][1,3]oxazole-2,6,8,14(1H,7H,9H)-tetrone;
methyl(5-(5-(3-(((3aS,4R,7S,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyl-2,6,8,14-tetraoxotetradecahydro-2H-oxacyclotetradecino[4,3-d][1,3]oxazol-11-yl)oxy)prop-1-ynyl)thien-2-yl)-2H-tetraazol-2-yl)acetate;
(3aS,4R,7S,9R,10R,11S,13R,15R,15aR)-11-((3-(5-(2-allyl-2H-tetraazol-5-yl)thien-2-yl)prop-2-ynyl)oxy)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyloctahydro-2H-oxacyclotetradecino[4,3-d][1,3]oxazole-2,6,8,14(1H,7H,9H)-tetrone;
(3aS,4R,7S,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyl-11-((3-(6-(2-methyl-2H-tetraazol-5-yl)pyridin-3-yl)prop-2-ynyl)oxy)octahydro-2H-oxacyclotetradecino[4,3-d][1,3]oxazole-2,6,8,14(1H,7H,9H)-tetrone;
(3aS,4R,7S,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyl-11-((3-(5-(2-(2-cyanoethyl)-2H-tetraazol-5-yl)thien-2-yl)prop-2-ynyl)oxy)octahydro-2H-oxacyclotetradecino[4,3-d][1,3]oxazole-2,6,8,14(1H,7H,9H)-tetrone;
(3aS,4R,7S,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyl-11-((3-(2-methyl-2H-tetraazol-5-yl)prop-2-ynyl)oxy)octahydro-2H-oxacyclotetradecino[4,3-d][1,3]oxazole-2,6,8,14(1H,7H,9H)-tetrone;
(3aS,4R,7S,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-7-fluoro-3a,7,9,11,13,15-hexamethyl-11-((3-(2-(2-methyl-2H-tetraazol-5-yl)-1,3-thiazol-5-yl)prop-2-ynyl)oxy)octahydro-2H-oxacyclotetradecino[4,3-d][1,3]oxazole-2,6,8,14(1H,7H,9H)-tetrone; or
(3aS,4R,7R,9R,10R,11S,13R,15R,15aR)-10-(((2S,3R,4S,6R)-4-(dimethylamino)-3-hydroxy-6-methyltetrahydro-2H-pyran-2-yl)oxy)-4-ethyl-3a,7,9,11,13,15-hexamethyl-11-((3-(2-(2-methyl-2H-tetraazol-5-yl)-1,3-thiazol-5-yl)prop-2-ynyl)oxy)octahydro-2H-oxacyclotetradecino[4,3-d][1,3]oxazole-2,6,8,14(1H,7H,9H)-tetrone.
